(19) United States
(12) Patent Application Publication
(10) Pub. No.: US 2010/0048729 A1
(43) Pub. Date: Feb. 25, 2010

(54) Composition based on xanthoxyline and its use in cosmetic

(75) Inventors: Marie Charveron, Toulouse (FR); Francoise Belaubre, Cugnaux (FR); Rene Belle, Saix (FR); Christophe Dunouau, Pin-Balama (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: PIERRE FABRE DERMO-COSMETIQUE, Boulogne (FR)

(21) Appl. No.: 12/310,960

(22) PCT Filed: Sep. 11, 2007
(86) PCT No.: PCT/FR2007/001467

§ 371 Date: Aug. 10, 2009

(30) Foreign Application Priority Data
Sep. 15, 2006 (FR) 0608129

(51) Int. Cl. A61K 31/12

(52) U.S. Cl. 514/689

(57) ABSTRACT

The present invention relates to the use of xanthoxyline, and of plant extracts containing it, in cosmetic compositions.

The present invention relates to the use of xanthoxyline, and of plant extracts containing it, for the treatment of cellulite.

Xanthoxyline, defined as 4,6-dimethoxy-2-hydroxyacetophenone, is also called brevifoline. It is a compound of natural origin. It is present in numerous plants, which are principally grouped into 2 botanical families, the Rutaceae, where it is found in the following plants: Melicope borbonica, Phebalium tuberculosum, Phebalium filifolium, and in the genus Zanthoxylum with Z. rhoifolium, Z. armatum, Z. bungeanum, Z. piperitum; the other family rich in xanthoxyline is that of the Euphorbiaceae with Croton nepetaefolium, Hippomane mancinella, Sapium sebiferum, Sebastiana schottiana, Euphorbia quinquecostata and Euphorbia fidjiana. It is also described in an Inuleae=Blumea balsamifera and an Asteraceae=Artemisia brevifolia.

Xanthoxyline has already been the subject of some evaluation studies. Its anti-fungal and anti-microbial properties have been investigated widely. Anti-fungal activity is moderate especially with respect to Candida albicans and Penicillium expansum (Simonsen H. T., Phytotherapy Research, July 2004, vol 18, n° 7, p 542-545) and with respect to pathogenic fungi such as Trichophyton (Pinheiro T. R. et al. Arzneimittel-Forschung, Dec 1999, vol 49, n° 12, p 1039-1043). Anti-bacterial activity is likewise mediocre with respect to gram-positive and gram-negative bacteria (Gonzaga-Wellington A. et al., Planta Medica, April 2003, vol 69, n° 4, p 371-374).

In therapeutics, xanthoxyline and also certain derivatives have been investigated mainly as anti-spasmodics (Cechinel-Filho V. et al., Journal of Pharmaceutical Sciences, April 1995, vol 84, n° 4, p 473-475; Calixto J. B. et al., Planta Medica, February 1990, vol 56, n° 1, p 31-35; Hashimoto et al., Planta Medica, 2001, vol 67 n° 2, p 179-81).

Xanthoxyline has also been the subject of protection in the therapeutic field as a modulator of cysteine protease activity (WO 9930699).

Surprisingly and unexpectedly, the Applicant has demonstrated a novel activity for xanthoxyline and plant extracts containing it in respect of inhibiting the differentiation of preadipocytes into adipocytes. That kind of activity is of particular interest in the cosmetic slimming field, since the differentiation of preadipocytes into adipocytes manifests itself in even greater excess fat in adipose tissue and especially in cellulitic tissue.

The present invention thus relates to the use of xanthoxyline for a slimming treatment; and for the prevention or treatment of excess adipose and cellulite.

In the context of the present invention, xanthoxyline may be obtained by chemical means or from a plant extract.

The present invention relates preferably to the use of plant extracts containing xanthoxyline: the plant extract advantageously originates from plants from the family of the Rutaceae: Melicope borbonica, Phebalium tuberculosum, Phebalium filifolium, and in the genus Zanthoxylum with Z. rhoifolium, Z. armatum, Z. bungeanum, Z. piperitum; the Euphorbiaceae: Croton nepetaefolium, Hippomane mancinella, Sapium sebiferum, Sebastiana schottiana, Euphorbia quinquecostata, Euphorbia fidjiana but also from Blumea balsamifera and Artemisia brevifolia and other plants that contain it. Preferably, the xanthoxyline originates from a Zanthoxylum bungeanum extract.

The plant extract is prepared according to conventional preparation steps known to the person skilled in the art.

The plant, preferably dried, is ground and then extracted with an organic solvent, which may be an alkane (pentane, hexane, heptane, octane, cyclohexane), an ether (tetrahydrofuran, dioxane, diethyl ether), an ester (ethyl acetate, isopropyl acetate), an alcohol (methanol, ethanol, propanol, isopropanol, butanol), a ketone (methyl ethyl ketone, dimethylketone, methyl isobutyl ketone), a halogenated hydrocarbon (chloroform, dichloromethane), water or a mixture of any miscible proportion of those solvents. Extraction is carried out in a plant/solvent ratio of between approximately 1/1 and approximately 1/20 inclusive and may be repeated from 2 to 3 times. The temperature of the extraction solvent may be equal to or higher than ambient temperature, and may reach the boiling temperature of the solvent employed. The period of contact between the plant and the solvent is between approximately 30 min and approximately 72 hours inclusive. Solid/liquid separation is then carried out, the plant being separated from the solvent by filtration or centrifugation.

The filtrate obtained may be either:
concentrated to dryness straightaway by complete evaporation of the extraction solvent to form the final extract,
maintained in liquid form in the extraction solvent, if that is compatible with its use. In that case, it can be concentrated to a greater or lesser extent by an evaporation step,
purified. The purification step can be carried out by techniques known to the person skilled in the art, such as liquid/liquid extraction between 2 non-miscible solvents, absorption on a support such as silica, an ion exchange resin, a non-polar support such as polystyrene, precipitation, crystallisation, sublimation. After purification, the extract may be dried by evaporating off the solvent then drying, but it can equally be dissolved in a solvent compatible with its use.

An extract obtained by extraction, solid/liquid separation and then drying contains a fraction by weight of xanthoxyline that is between 0.1 and 30 g inclusive per 100 g of dry material in the extract, and preferably between 1 and 15 g inclusive per 100 g of dry material in the extract. If the extract is maintained in solution, the content of dry material in the liquid extract is between 0.1 g and 80 g inclusive per 100 ml of liquid extract according to the concentration carried out. The content of xanthoxyline may be expressed in accordance with the dry material present in the liquid extract, and will then be between 0.1 and 30 g inclusive per 100 g of that dry material, preferably between 1 and 15 g inclusive per 100 g of dry material.

The purification techniques allow extracts rich in xanthoxyline to be obtained in which the fraction by weight of xanthoxyline is greater than 30 g per 100 g of dry material in the extract according to the techniques undertaken. Advantageously, the said fraction by weight of xanthoxyline is between 50 g and 100 g inclusive per 100 g of dry material in the enriched extract.

The chemical synthesis of xanthoxyline is known to the person skilled in the art. It is described, for example, in Hunan Huagong: 1999: vol 29(6), P 27-28, ISSN: 1005-8435.

The present invention relates also to cosmetic compositions containing xanthoxyline as a slimming active ingredient. In the context of the present invention, xanthoxyline acts as active ingredient in the prevention or treatment of excess adipose and cellulite.

One of the aspects of the present invention concerns new cosmetic slimming compositions containing at least xanthoxyline in association with a suitable cosmetic carrier.

The suitable cosmetic carrier can be selected, inter alia, from diluents, dispersants, gelling agents, gums, resins, oily agents, fatty alcohols, waxes, preservatives, colorants, absorption-promoting agents, flavourings and perfumes, used alone or in a mixture.

The said cosmetic slimming composition is preferably in topical or oral form. The topical form is advantageously a gel, a spray, a cream, a cream gel, an ointment, a milk or a lotion.

The composition may also be in an oral form, such as in the form of a tablet, a gelatin capsule or a powder for drinkable suspensions.

Advantageously, the acceptable cosmetic excipient is suitable for topical application of the said composition to the zone of the skin to be treated, or for oral administration of the said composition.

Even more advantageously, the person skilled in the art will adapt an acceptable cosmetic excipient for the topical application of the said composition to the zone of the skin to be treated. The cosmetic carrier is thus conventionally selected by the person skilled in the art to promote passage of the xanthoxylene applied by the cutaneous route across the cutaneous barrier and to the adipocytes.

The selection and/or quantity of the ingredient or ingredients of the said carrier will also be determined by the tolerance and the specific needs of the skin to which the composition is to be applied as well as by the properties and the desired consistency of the composition according to the present invention.

The slimming effectiveness of the composition according to the invention is reflected in:
a centimetre loss especially at the thighs and/or the hips and/or the waist;
a reduction in the thickness of the adipose tissue, especially at the thighs and/or the hips and/or the waist;
and/or a reduction in the volume of the thighs.

The amount of xanthoxyline introduced into the composition according to the invention is between approximately 0.1 mg and approximately 100 mg inclusive per 100 g of composition, preferably between approximately 0.5 mg and approximately 50 mg inclusive per 100 g of composition, even more preferably between approximately 1 mg and approximately 20 mg inclusive.

The xanthoxyline of the said compositions can be obtained by chemical means or from a plant extract. Preferably, the extract originates from plants from the family of the Rutaceae: Melicope borbonica, Phebalium tuberculosum, Phebalium filifolium, and in the genus Zanthoxylum with Z. rhoifolium, Z. armatum, Z. bungeanum, Z. piperitum, the Euphorbiaceae: Croton nepetaefolium, Hippomane mancinella, Sapium sebiferum, Sebastiana schottiana, Euphorbia quinquecostata, Euphorbia fidjiana, but also from Blumea balsamifera and Artemisia brevifolia and other plants that contain it. Preferably, xanthoxyline is extracted from Zanthoxylum bungeanum.

The present invention relates to cosmetic slimming compositions based on xanthoxyline as the slimming active ingredient. Preferably, the said compositions in addition contain at least a second slimming active ingredient. That second active ingredient likewise allows excess adipose and cellulite to be controlled.

The other cosmetic slimming agent or agents that can be added to the present composition are known to the person skilled in the art, who will be able to adjust the relative proportions of each constituent of the composition to optimise the effectiveness of the said composition.

Advantageously, there may be mentioned by way of example and without implying any limitation, slimming active ingredients selected from the group formed by caffeine and its salts, phloridzine, forskoline, hesperidine methyl chalcone, guarana, mate, extracts of mouse-ear hawkweed, of ruscus, of ivy, of apple tree branches and of Coleus forskohlii, used alone or in a mixture.

Finally, the present invention relates also to a method for the cosmetic treatment of the skin to prevent or reduce excess adipose and cellulite, which comprises the administration of a composition based on xanthoxyline topically, to the zone of the skin requiring such treatment, or orally. The topical form is advantageously selected from the group consisting of a gel, a spray, a cream, a cream gel, an ointment, a milk or a lotion. The oral form is advantageously selected from the group consisting of tablets, gelatin capsules and powders for drinkable suspensions.

The following preparations and compositions are mentioned by way of illustrative examples, without implying any limitation.

### EXAMPLES OF THE PREPARATION OF THE PLANT EXTRACT

### Example 1

preparation of a Sebastiana schottiana Root Extract.

5 kg of ground dry roots of Sebastiana schottiana are extracted in 2 extractions with 30 ml and 20 l of 95% ethanol at reflux.

After concentrating the combined filtrates to approximately 30 litres, 10 litres of absolute alcohol are added, chilling at +4° C. is carried out overnight, and rapid filtration in the cold under pressure is carried out to remove any waxes that may be present. The fluid extract obtained has a hydroxy-methoxy-acetophenone content. Its xanthoxyline content is between 0.1 and 5% in relation to the dry material.

### Example 2

Preparation of an Extract of Fruit Pericarps of Zanthoxylum bungeanum

100 kg of fruit pericarps of Zanthoxylum bungeanum are ground and twice extracted at reflux with 500 l of heptane.

After concentration to a soft extract, 1000 litres of absolute alcohol are added. Concentration is carried out until residual heptane has been completely eliminated azeotropically, and the concentrate is chilled at +4° overnight. The waxy flocculate is removed by filtration under pressure and the fluid extract is standardised to 1 g/l of xanthoxyline.

### Example 3

Preparation of Xanthoxyline in Crystallised Form from an Extract of Fruit Pericarps of Zanthoxylum bungeanum

100 kg of ground fruit pericarps of Zanthoxylum bungeanum are extracted at reflux with 500 litres of 90% ethanol.

The filtrate is concentrated in vacuo to approximately 100 litres and the active ingredient is enriched by liquid/liquid extraction with 3×20 litres of heptane.

The combined heptane phases are concentrated to dryness. The crude xanthoxyline is redissolved in 5 litres of warm absolute ethanol. That solution, cooled, is subjected to chilling at +2° C. for 24 hours and filtered cold very rapidly. By concentration to approximately 1 litre and resting at ambient temperature, approximately 1 kg of crystallised xanthoxyline is obtained, which is separated by filtration and washing over a filter with 200 ml of cold absolute ethanol, followed by drying in vacuo at ambient temperature. Its purity is between 95 and 100%.

### Example 4

Preparation of Xanthoxyline in Crystallised Form from an Extract of Fruit Pericarps of Zanthoxylum bungeanum

It is likewise possible to obtain the active ingredient by direct sublimation from ground pericarps heated in a boiling bain-marie under a vacuum of 15 mm Hg. The xanthoxyline is collected crystallised on a cold condenser (10° C.), its purity being between 95 and 100%.

### COSMETIC COMPOSITION EXAMPLE

Slimming cream gel n° 1:

| | |
|---|---|
| Xanthoxyline | 0.001 to 1% |
| Hesperidine methyl chalcone | 0.3 to 1% |
| Caffeine base | 1 to 5% |
| Carboxylic caffeic acid | 2% |
| Apple tree branch extract | 0.1 to 5% |
| Capric/caprylic triglycerides | 20% |
| 95% Ethanol | 10% |
| Triethanolamine | 0.3% |
| Carboxyvinyl polymer | 0.3% |
| Perfume | q.s. |
| Preservatives colorants | |
| Potable water ad 100 g | |

Slimming cream gel n° 2:

| | |
|---|---|
| Xanthoxyline | 0.001 to 0.02% |
| Hesperidine methyl chalcone | 0.1 to 5% |
| Caffeine base | 1 to 5% |
| Carboxylic caffeic acid | 0.1 to 5% |
| Apple tree branch extract | 0.1 to 5% |
| Capric/caprylic triglycerides | 15 to 25% |
| 95% Ethanol | 10% |
| Triethanolamine | 0.3% |
| Carboxyvinyl polymer | 0.3% |
| Perfume | q.s. |
| Preservatives colorants | |
| Potable water ad 100 g | |

Slimming spray for obstinate zones:

| | |
|---|---|
| Zanthoxylum bungeanum extract | 1 to 5% |
| Carboxylic caffeic acid | 5% |
| Apple tree branch extracts | 0.1 to 10% |
| Guanidine carbonate | 0.5% |
| Fluid extract of ruscus | 1% |
| Polyvinyl alcohol | 0.2% |
| P.E.G. 400 | 1% |
| 95% alcohol | 30% |
| Potable water ad 100 g | |

Decongesting slimming massage cream:

| | |
|---|---|
| Zanthoxylum bungeanum extract | 1 to 10% |
| Apple tree branch extracts | 0.1 to 10% |
| Mouse-ear hawkweed extract | 1 to 5% |
| 3-nicotinol caffeine carboxylate | 1 to 20% |
| Vitamin E acetate | 0.5% |
| Massage excipient ad 100 g | |

### PHARMACOLOGICAL EVALUATION

In Vitro Study of the Action of a Dry Extract of Zanthoxylum bungeanum and of Xanthoxyline on Adipocyte Differentiation

Adipocyte differentiation (differentiation of preadipocytes into adipocytes) is a complex biological phenomenon regulated at the molecular level by the activation of specific genes leading to a particular adipocyte phenotype characterised by the accumulation of lipid droplets.

Adipocytes are capable of hydrolysing triglycerides and thereby releasing fatty acids and glycerol, a lipolysis tracer.

The Applicant has studied on the one hand the effect of a dry extract of Zanthoxylum bungeanum, and on the other hand the effect of xanthoxyline, on the process of adipocyte differentiation and proliferation. The reference used for this study is the cytokine TNFα (known to inhibit the accumulation of triglycerides) in the adipocyte differentiation model over a long culture period (Journal of Clinical Endocrinology and Metabolism: PETRUSCHKE T., 1993, 76(3): 742-747).

Experimental protocol:

3T3F442A prepadipocytes are capable of differentiating into adipocytes in the presence of insulin, 3-isobutyl-1-methylxanthine and dexamethasone (the Journal of Pharmacology and Experimental Therapeutics: RIVAL Y., 2004, 311(2): 467-475). The differentiation is accompanied by an accumulation of intracellular triglyceride droplets revealed by the reagent Adipored Assay Reagent (CAMBREX, PT-7009).

3T3F442A cells are cultured in DMEM medium (GIBCOBRL, ref 32430-027) containing 10% foetal calf serum at 37° C., 5% CO₂ in a humid atmosphere. They are seeded at 5000 cells per well (96-well plate). Two days after obtaining a confluent cell carpet, the cells are put into differentiation condition in a medium containing 1.7 μM of insulin, 0.5 M of 3-isobutyl-1-methylxanthine and 1 μM of dexamethasone, in the presence or absence of the test products, for 7 days. The test products are, on the one hand, the dry form of the alcoholic extract of Zanthoxylum bungeanum adjusted to 1 g/l of xanthoxyline prepared in accordance with Example 2 compared with human TNFα (R&D, 210-TA); and on the other hand xanthoxyline obtained in accordance with Example 3 compared with human TNFα (R&D, 210-TA).

The visualisation and quantification of intracellular lipid vacuoles in the differentiated cells are analysed using Adipored reagent, a fluorescent colorant specific to triglycerides (neutral and amphiphilic lipids) (Journal of Cell Biology: GREENSPAN P., 1985, 100: 965-973).

The culture medium is removed and the cells are rinsed with phosphate buffer D-PBS (GIBCOBRL, ref 21300-058). Adipored reagent is then added. After incubation for 10 minutes at ambient temperature, the fluorescence is measured (excitation 485 nm, emission 530 nm). A histological analysis using a confocal Laser microscope (ZEISS LSM 410 Invert Laser scan Microscope) is also carried out at 543 nm.

Results

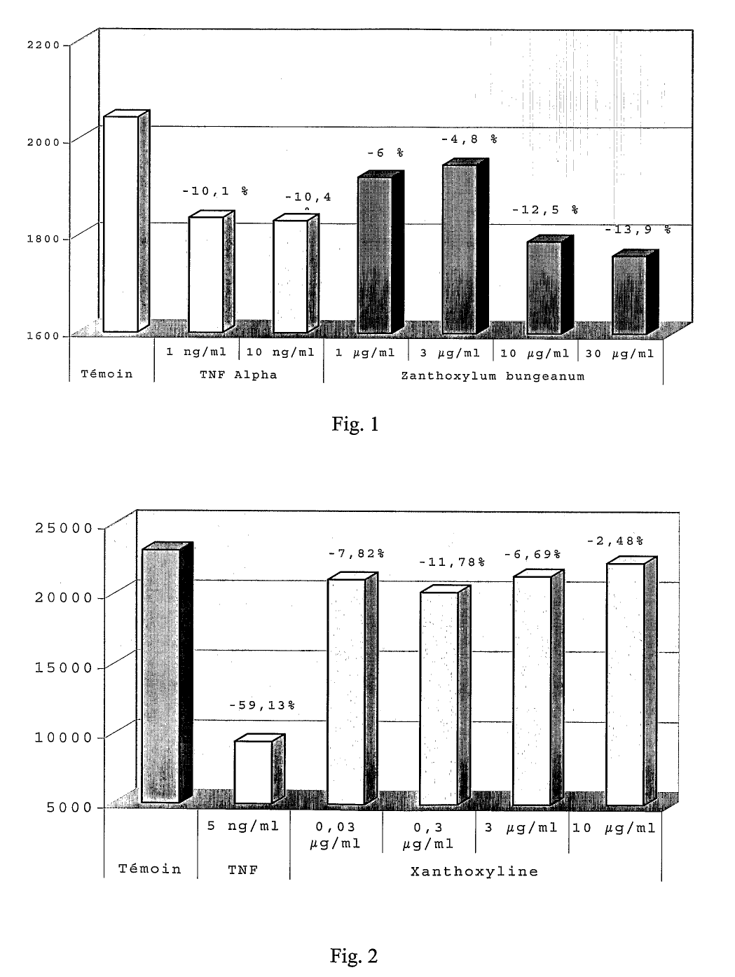

FIGS. 1 and 2 represent the accumulation of triglycerides in the differentiated cells (expressed in random unit of Adipored) when the differentiation has been induced respectively by different concentrations of Zanthoxylum bungeanum extract and of xanthoxyline.

Seven days after treatment with a mixture of insulin, 3-isobutyl-1-methylxanthine and dexamethasone, the 3T3F442A preadipocytes differentiate into adipocytes. Their morphology changes, and the cells become round and accumulate triglycerides, which are visualised by the colorant Adipored using the confocal microscope.

Differentiation of the cell line was induced in the presence of the various test products and was assessed by the quantification of triglyceride accumulation.

Zanthoxylum bungeanum extract:

The results are shown in FIG. 1: the first measurement corresponds to the control differentiated in the absence of the test product, then the two series of measurements represented correspond respectively to TNFα at 1 and 10 ng/ml and to Zanthoxylum bungeanum extract at 1, 3, 10 and 30 μg/ml.

It will be observed that Zanthoxylum bungeanum extract inhibits the accumulation of triglycerides significantly at 10 and 30 μg/ml.

Xanthoxyline:

The results are shown in FIG. 2: the first measurement corresponds to the control differentiated in the absence of the test product, the second measurement corresponds to TNFα at 5 ng/ml and finally the following series of measurements correspond respectively to xanthoxyline concentrations of 10, 3, 0.3 and 0.03 μg/ml.

It will be observed that xanthoxyline exhibits optimum activity (12% inhibition) at a concentration of 0.3 μg/ml.

### STUDY OF THE EFFECTIVENESS OF A COMPOSITION BASED ON XANTHOXYLINE

The clinical study presented hereinbelow was an open study carried out with 61 women aged from 25 to 45 having a body mass index of between 22 and 26 kg/m⁻² inclusive.

The study concerns the cream gel composition n° 2 given as an example hereinabove. The product was applied once per day in the morning for 28 days to the hips, the buttocks; the abdomen and randomly to one thigh. On day 28, the sample group was 60 women.

The starting point for this effectiveness study is noted as d0 (prior to the first application).

Various evaluation techniques were used: centimetre measurements, measurements of the thickness of the adipose tissue and measurements of the volume of the thighs.

The Figures hereinbelow show the following:

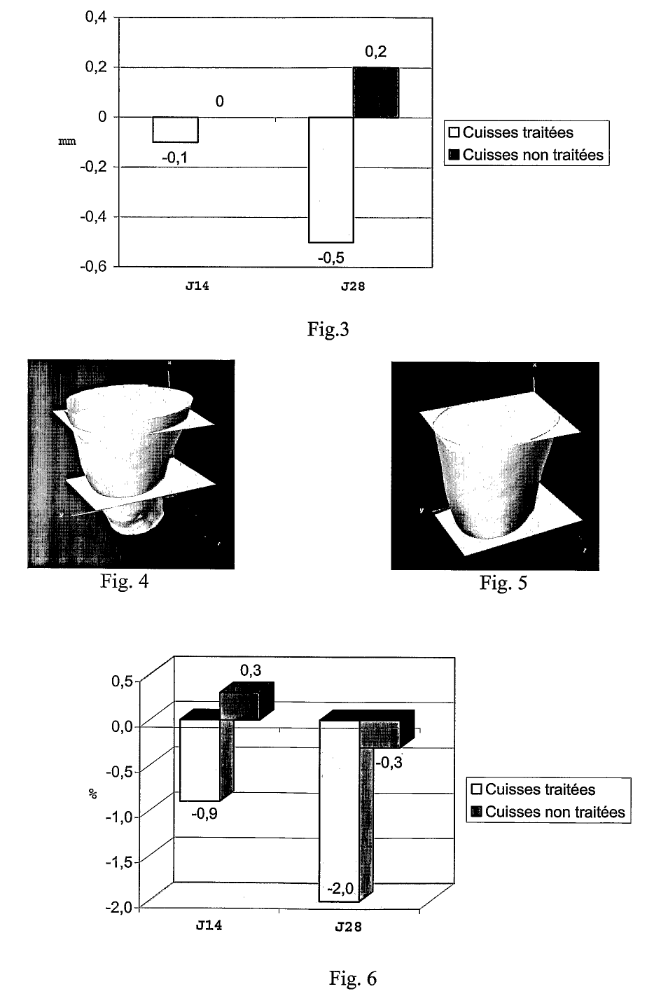

FIG. 3: change in the thickness (mm) of the adipose tissue of the thighs over the sample group as a whole;

FIGS. 4 and 5: horizontal reference planes determining the measured volume of the thighs;

FIG. 6: change (%) in the volume of the thighs over the sample group as a whole.

1. Centimetre measurement

Protocol:

The centimetre measurements are taken at:
the two thighs
the waist (navel level)
the hips

The centimetre measurements are taken after locating each site using a graduated vertical rail fitted with a laser, which determines the height in relation to the ground and enables correct vertical positioning.

After drawing 4 horizontal marks in pencil on the circumference of each site, the measurement is obtained by means of a flexible measure applied precisely, and without compression, beneath that line.

Results:

The results of the centimetre measurements for the various zones are compiled in Table 1 in terms of the results on d14 (after 14 days of treatment) and on d28 (after 28 days of treatment).

The following are recorded:
thigh measurement=measurement of treated thigh on d14 or d28 minus thigh measurement on d0
The mean of those values for the subjects of the study as a whole is given, as well as the maximum value obtained.
the difference between the measurement of the treated thigh and the measurement of the untreated thigh on d14 or d28
The maximum difference between the treated thigh and the untreated thigh is given.
hip measurement=measurement of the treated hips on d14 or d28 minus hip measurement on d0
The mean of those values for the subjects of the study as a whole is given, as well as the maximum value obtained.
waist measurement=waist measurement on d14 or d28 minus waist measurement on d0.
The mean of those values for the subjects of the study as a whole is given, as well as the maximum value obtained.

TABLE 1
results of centimetre measurements on d14 and d28

Composition
according to the
present invention

| | d14 | d28 |
|---|---|---|
| | Mean | Mean |
| Measurements in cm | (n = 61) | (n = 60) |
| Thigh measurement | −0.5 | −0.8 |
| Max. | −1.5 | −2.2 |
| (measurement of treated thigh) minus | −0.3 | −0.6 |
| (measurement of untreated thigh) | | |
| Max. | −1.1 | −1.8 |
| Thigh measurement | −0.7 | −1.0 |
| Max. | −3.3 | −3.8 |
| Waist measurement | −0.6 | −0.8 |
| Max. | −2.9 | −2.5 |

2. Measurements of the thickness of the adipose tissue of the thighs (by echography)

Protocol:

The equipment used is an echograph, type EUB 415—HITACHI—JAPAN, fitted with a linear probe operating at a frequency of 7.5 MHz.

The ultrasound imagery consists in carrying out a cartography of the differences in impedance of the tissues in question with regard to ultrasonic waves. Following the emission of a beam of waves by a probe, the receipt of the echoes and the value of the intensity thereof in association with the absorption thereof by the various tissues allows an image to be constructed in the plane of the beam.

Three images are obtained in succession at the cutaneous marker of each measurement site.

On each exposure, three thickness measurements are carried out.

The measurements are carried out at the apex of the cellulite bulge located on the external face of each thigh.

The value retained corresponds to the mean of the three measurements taken.

Results:

The results after treatment for 14 and 28 days (written, respectively, as d14 and d28) are presented in FIG. 3: the results express the difference (in mm) between the thickness of the adipose tissue on d14 or d28 and the thickness of the adipose tissue of the same thigh on d0.

The measurements obtained for the treated thighs [symbol] were compared with the same measurements for the untreated thighs (▪).

After 14 days of treatment: a mean reduction in the thickness of the adipose tissue of 0.1 mm is recorded for n=29 subjects.

After 28 days of treatment: a significant mean reduction in the thickness of the adipose tissue of 0.5 mm is recorded, that is, a reduction of 1.6% in the thickness of the adipose tissue, for n=26 subjects.

3. Thigh volume measurements (<<in vivo>> fringe projection technique)

Protocol:

The fringe projection technique, based on the principal of optical triangulation with patterned light, allows acquisitions in three dimensions of the volume of each thigh. It allows the change in volume of the thighs to be visualised and quantified.

This system comprises a measurement sensor combining a halogen projector coupled to a high-resolution 768×576 pixel CCD camera calibrated for a measurement field of 240 mm—MicroTop system (EoTech, France)—interfaced with Optocat acquisition software (EoTech, France). The mean resolution is approximately 150 μm in 3 spatial directions (x, y, z).

This system allows a network of lines to be projected onto the zone to be measured; the deformations of those lines are recorded by the camera with a view to data processing. A series of 6 acquisitions is carried out at different incidences (0°, 60°, 120°, 180°, 240°, and 300°) relative to the centre of rotation of the zone being measured. The 6 acquisitions are automatically rescaled by geometric realignment, allowing reconstruction of the surface of the thigh. The volume is determined after defining 2 horizontal planes of reference (FIGS. 4 and 5).

Each plane is defined after superposition of the 3D reconstructions of a given thigh at different times of the kinetics in order to obtain a reproducible repositioning. Those horizontal planes determine the measured volume of the thighs.

Results:

The results after treatment for 14 and 28 days (written as d14 and d28) are presented in FIG. 6: they express the change (in %) in the volume of the thigh on d14 or d28 compared with the volume of the same thigh on d0.

The measurements obtained for the treated thighs [symbol] were compared with the same measurements for the untreated thighs (▪).

After 14 days of treatment: a significant reduction in the volume of the treated thighs of 0.9% compared with d0 is observed in n=29 subjects. The difference (treated thigh minus untreated thigh) is also significant.

After 28 days of treatment: a signification reduction in the volume of the treated thighs of 2% compared with dO is observed in n=27 subjects. The difference (treated thigh minus untreated thigh) is also significant.

### CONCLUSION OF THE EFFECTIVENESS STUDY

This study has allowed the slimming effectiveness of a composition according to the present invention to be demonstrated.

The percentages of the subjects that respond are very large. At the end of 28 days, by comparison with d0 95% of the subjects have a centimetre loss at the treated thigh; the same applies to 90% of the subjects in respect of the hips.

From 14 days, the differences compared with dO in terms of the treated thigh, the hips and the waist are significant. At 28 days those results are accentuated.

The difference (treated thigh minus untreated thigh), which is more representative of the effectiveness, is also significant from 14 days.

The significant results of the echography and the fringe projection confirm the results obtained by the centimetre measurement.

1-19. (canceled)

20. A method for cosmetic treatment comprising administering xanthoxyline to a subject in an amount effective for slimming.

21. The method of claim 20, wherein the administration of xanthoxyline prevents or treats excess adipose and cellulite.

22. The method of claim 20, wherein the xanthoxyline is comprised in a plant extract or is obtained from a plant extract.

23. The method of claim 22, wherein the plant extract is derived from plants of the family Rutaceae selected from Melicope borbonica; Phebalium tuberculosum; Phebalium filifolium; from the genus Zanthoxylum selected from Z. rhoifolium; Z. armatum; Z. bungeanum; Z. piperitum; from the Euphorbiaceae selected from Croton nepetaefolium; Hippomane mancinella; Sapium sebiferum; Sebastiana schottiana; Euphorbia quinquecostata; Euphorbia fidjiana; and from Blumea balsamifera and Artemisia brevifolia.

24. The method of claim 22, wherein the plant extract is derived from Zanthoxylum bungeanum.

25. The method of claim 22, wherein the fraction by weight of xanthoxyline is between 0.1 and 30 g, inclusive, per 100 g of dry extract.

26. The method of claim 22, wherein the plant extract is enriched with xanthoxyline, comprising a fraction by weight of xanthoxyline greater than 30 g per 100 g of dry extract.

27. The method of claim 20, wherein the xanthoxyline is administered in an oral form, or is administered topically to a zone of skin requiring the cosmetic treatment.

28. The method of claim 27, wherein the xanthoxyline is administered in a topical form selected from a gel, a spray, a cream, a cream gel, an ointment, a milk and a lotion.

29. The method of claim 27, wherein the xanthoxyline is administered in an oral form selected from tablets, gelatin capsules and powders for drinkable suspensions.

30. A method of preventing or reducing excess adipose and/or cellulite comprising administering xanthoxyline to a subject, wherein the xanthoxyline is administered in an oral form, or is administered topically to a zone of skin requiring treatment or prevention of excess adipose and/or cellulite.

31. A cosmetic slimming composition comprising xanthoxyline and suitable carriers and/or excipients.

32. The cosmetic slimming composition of claim 31, further comprising at least one other slimming active agent.

33. The cosmetic slimming composition of claim 32, wherein the other slimming active agent is selected from caffeine and its salts, phloridzine, forskoline, hesperidine methyl chalcone, guarana, mate, extracts of mouse-ear hawkweed, extracts of ruscus, extracts of ivy, extracts of apple tree branches, extracts of Coleus forskohlii, and mixtures thereof.

34. The cosmetic slimming composition of claim 31, wherein the xanthoxyline is comprised in a plant extract or is obtained from a plant extract.

35. The cosmetic slimming composition of claim 34, wherein the plant extract is derived from plants of the family Rutaceae selected from Melicope borbonica; Phebalium tuberculosum; Phebalium filifolium; from the genus Zanthoxylum selected from Z. rhoifolium; Z. armatum; Z. bungeanum; Z. piperitum; from the Euphorbiaceae selected from Croton nepetaefolium; Hippomane mancinella; Sapium sebiferum; Sebastiana schottiana; Euphorbia quinquecostata; Euphorbia fidjiana; and from Blumea balsamifera and Artemisia brevifolia.

36. The cosmetic slimming composition of claim 34, wherein the plant extract is derived from Zanthoxylum bungeanum.

37. The cosmetic slimming composition of claim 31, which is in an oral form or a topical form.

38. The cosmetic slimming composition of claim 37, wherein the topical form is selected from a gel, a spray, a cream, a cream gel, an ointment, a milk and a lotion.

39. The cosmetic slimming composition of claim 37, wherein the oral form is selected from tablets, gelatin capsules and powders for drinkable suspensions.
